(19) United States
(12) Patent Application Publication
(10) Pub. No.: US 2008/0208227 A1
(43) Pub. Date: Aug. 28, 2008

(54) Self-contained dissector/harvester device

(75) Inventors: Randal James Kadykowski, South Lyon, MI (US); Lyne Madeleine Charron-Keller, Brighton, MI (US)

Correspondence Address: TERUMO CARDIOVASCULAR SYSTEMS CORPORATION;ATTN: GAEL DIANE TISACK, 6200 JACKSON ROAD, ANN ARBOR, MI, 48103, US

(73) Assignee: Terumo Cardiovascular Systems Corporation

(21) Appl. No.: 11/710,144

(22) Filed: Feb. 23, 2007

(51) Int. Cl. A61B 17/22

(52) U.S. Cl. 606/159; 606/27

(57) ABSTRACT

A self-contained device for dissecting and/or harvesting a vessel includes a self-contained light source and a self-contained imaging system positioned within the sheath.

### CROSS-REFERENCE TO RELATED APPLICATIONS

Not applicable.

### STATEMENT REGARDING SPONSORED RESEARCH

Not applicable.

### BACKGROUND OF THE INVENTION

The present invention relates to the harvesting of blood vessels and, more particularly, to a method and apparatus for dissection and removal of sections of blood vessels.

The harvested vessels are used in many surgical procedures, including use as a coronary artery bypass graft, or in other cardiovascular procedures. As one example, in vascular and cardiovascular procedures, a blood vessel or vessel section, such as an artery or vein, is “harvested” (i.e., removed) from its natural location in a patient's body and is used elsewhere in the body. In coronary artery bypass grafting surgery, for example, the harvested blood vessel is used to form a bypass between an arterial blood source and the coronary artery that is to be bypassed. Among the preferred sources for the vessels to be used as the bypass graft are the saphenous vein in the leg and the radial artery in the arm.

Endoscopic surgical procedures for harvesting a section of a blood vessel (e.g., the saphenous vein) subcutaneously have been developed in order to avoid disadvantages and potential complications of harvesting of the blood vessel. In the past, the harvesting was done through a continuous incision (e.g., along the leg) that exposed the full length of the desired vein section. The continuous incision had been necessary in order to provide adequate exposure for visualizing the vein and for introducing the surgical instruments to sever, cauterize and ligate the tissue and side branches of the vessel.

A more recent development has been a minimally-invasive technique that employs a small incision for locating the desired vessel and for introducing one or more endoscopic devices into the small incision.

Commercially available products for performing the endoscopic blood vessel harvesting procedure include a number of separate devices that are each connected to remote or outside devices. Current devices use an insufflation device having plastic tubing to supply air or CO₂ device to insufflate the subcutaneous area, an endoscope having a camera and light cables in order to visualize both the dissection and harvesting procedures, and a harvester and/or dissector device having electrical or other lines to supply the dissecting and/or cauterizing of the vessel. In certain instances, the combination of wires, plastic tubing and fittings can be bulky and cumbersome for the person performing the vessel harvesting.

It would be desirable to have a dissector/harvester device that is more compact and does not require many external wires, cables or tubes.

### SUMMARY OF THE INVENTION

In one aspect, there is provided a self-contained device for dissecting and/or harvesting a vessel and severing any branches extending therefrom The self-contained dissecting/harvesting device has a sheath for insertion in a body through a cut skin portion.

In one particular aspect, positioned within the sheath are a self-contained light source, a self-contained imaging system, and a severing device.

In certain embodiments, the severing device comprises a tip in an axial position over a distal end of the sheath, the tip being configured to be axially movable between an open position and a closed position configured to dissect the vessel and branch upon movement to the closed position.

In certain embodiments, the self-contained light source and the self-contained imaging system are removable from the device. Also, when present, the self-contained insufflation device can be removable from the device.

In certain embodiments, both the distal end of the sheath and a proximal end of the tip have at least one generally mating surface whereby, when the tip is adjacent to the sheath, the generally mating surfaces are configured to secure the vessel being harvested while a vessel branch is being severed. The tip and the sheath can include electrodes configured for being electrically energized to sever and cauterize a vessel branch.

In certain embodiments, the self-contained dissector/harvester device further includes a self-contained insufflation device to supply a gas subcutaneously to an area adjacent to the vessel.

In another aspect, there is provided a method of severing branches from a vessel during harvesting from a body by a self-contained dissector/harvester device which includes: inserting a distal end of the self-contained dissector/harvester device into the body alongside the vessel to form a cavity substantially surrounding the vessel; activating the self-contained light source to at least illuminate a portion of the branch; activating the self-contained imaging system to view at least a portion of the illuminated branch; and, moving the severing device to a position substantially adjacent to the branch and activating the severing device, whereby the vessel and branch are severed from the vessel. In certain embodiments, the method further includes supplying a gas subcutaneously to an area adjacent to the vessel to be dissected and harvested.

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 is a structure diagram, partially in phantom, showing a handle portion and a tip or distal end of one embodiment of a self-contained dissector/harvester device in an open position.

FIG. 2 is a structure diagram, similar to the view in FIG. 1, showing one embodiment of the self-contained dissector/harvester device in a closed, or grasping, position on a vessel.

FIG. 3 is a structure diagram, partially in phantom and partially in cross-section, showing one embodiment of the distal end of a self-contained dissector/harvester device.

FIG. 4 is a structure diagram of one embodiment of a portion of the distal end of the self-contained dissector/harvester device showing a self-contained severing device.

FIG. 5 is a structure diagram of one embodiment of a portion of a self-contained imaging system within the distal end of the self-contained dissector/harvester device.

FIG. 6 is a structure diagram, partially in cross-section and partially in phantom, of a self-contained imaging system having an external free-standing insufflation device tethered to the self-contained dissector/harvester device.

FIG. 7 is a structure diagram, partially in cross-section and partially in phantom, of a self-contained imaging system having an externally mounted insufflation device connected to a sheath of the self-contained dissector/harvester device.

### DETAILED DESCRIPTION OF THE PREFERRED EMBODIMENT(S)

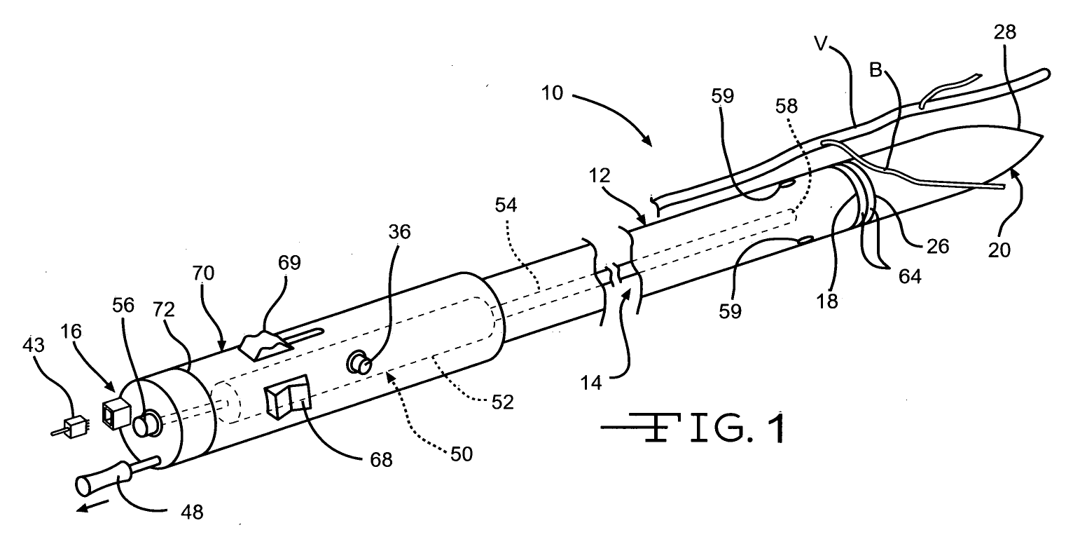

FIG. 1 is a structure diagram of one embodiment of a self-contained dissector/harvester device 10 for dissecting and/or harvesting a vessel V and for severing branches B from the vessel. The dissector/harvester device 10 includes an elongated sheath 12 for at least partial insertion in a body through a cut skin portion. The sheath 12 defines an inner space 14 and has a proximal end 16 and a distal end 18.

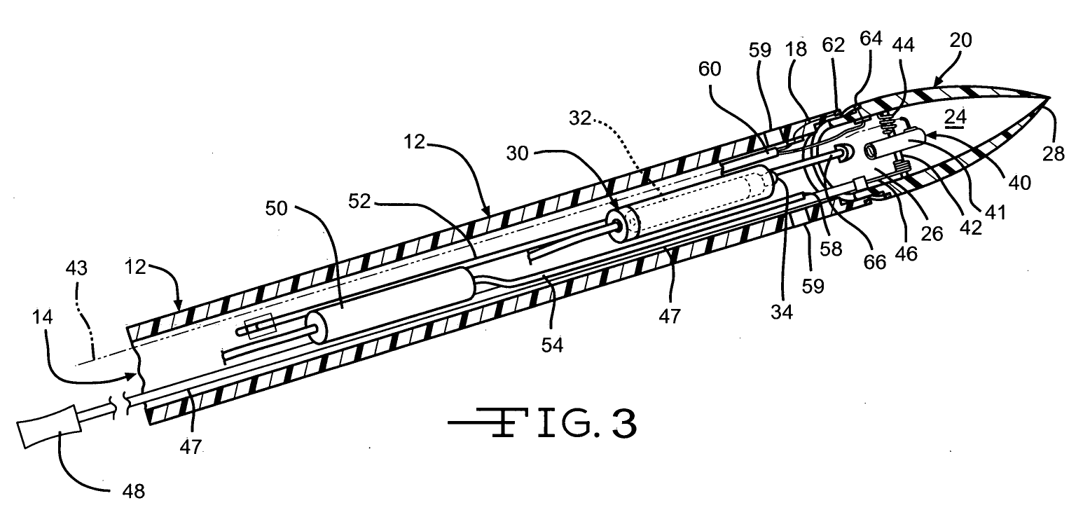

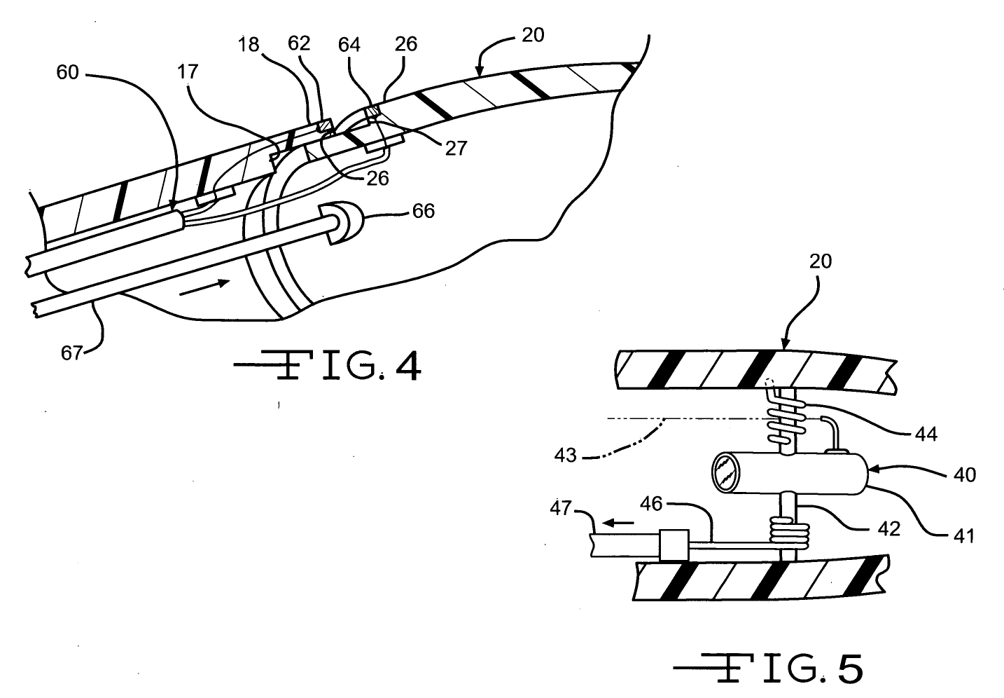

A tip 20 is axially positioned over the distal end 18 of the sheath 12. At least a portion of the dissector tip 20 is transparent in order to allow visualization of the vessel V and the surrounding tissue, as further explained below. As best seen in FIG. 3, the tip 20 defines an inner space 24 and has an open proximal end 26 and a closed distal end 28. The tip 20 is axially movable between an open position and a closed position. When the tip 20 is in the closed position, the tip proximal end 26 is adjacent to the sheath distal end 18. The distal end 28 of the tip 20 can have a conical or other tapered shape to aid in the harvesting of a vessel, as will further be explained below. In certain embodiments, as best seen in FIG. 4, the proximal end 26 of the tip 20 can have a stepped edge 27 that allows the proximal end 26 to be co-axially aligned within the distal end 18 of the sheath 12. Similarly, the distal end 18 can have a stepped edge 17 that allows the stepped edge 27 to be slidably received therein.

Referring again to FIG. 3, the self-contained dissector/harvester device 10 includes a self-contained light source 30 that is axially positioned within inner space 14 of the sheath 12. The light source 30 includes a power supply 32 and a light 34. In certain non-limiting embodiments, the light 34 can be a LED or fiber-optic device. Also, in certain embodiments, the power supply 32 can be a suitable battery-type power source that can be remotely controlled to be in an “on” or illuminating mode, or in an “off” or non-illuminating mode. In certain embodiments, the light source is slidably positioned within the inner space 14 of the sheath 12 and can be removed from the sheath 12 if the power supply 30 must be replaced. In certain embodiments, as schematically illustrated in FIG. 1, the power supply 30 is connected to a switch mechanism 36 to control the light 34.

The self-contained dissector/harvester device 10 also includes a self-contained imaging system 40 that is axially positioned within inner space 24 of the tip 20. It is to be understood, that in other embodiments, the self-contained imaging system 40 can be axially positioned within the inner space 14 of the sheath 12 is a spaced apart relationship to the tip 20.

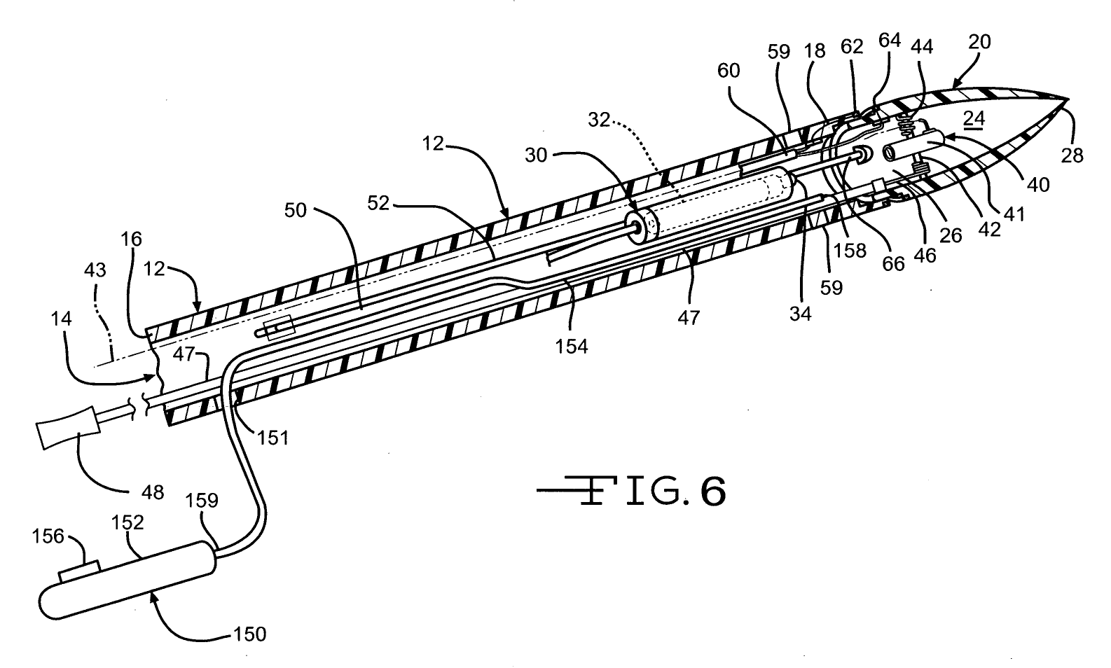

In the embodiment shown in FIGS. 3 and 5, the imaging system 40 is positioned within the tip 20. The imaging system 40 includes a suitable image receiving device 41 that converts images into signals for transmission, recording and/or storage, and/or takes photographs of such images; for ease of explanation herein such device is generally referred to as a camera.

The camera 41 that is pivotably mounted on a support 42 and can be oriented in a first, or proximal-viewing, direction toward the light source 30 or in a second, or distal-viewing, direction toward the tip 20. In one non-limiting embodiment shown in the FIGURES herein, the self-contained imaging system 40 can include, for example, a spring mechanism 44 axially mounted on the support 42. The spring mechanism 44 can include a tangentially extending end 46 that is operatively connected to a longitudinally extending member 47 which, in turn, is connected to a handle 48 for pivotably moving the camera 41 between the first and second directions.

It should be understood, that in other embodiments, other suitable mechanisms can be used for directing the movement of the camera 41 and/or for receiving the images from the imaging system 40. For example, in the embodiment shown in FIG. 1, the camera 41 is operatively connected to a suitable cable 43. The cable 43 can be connected to a suitable viewing monitor (not shown). In other embodiments, the camera 41 can be a wireless device that transmits images.

In certain embodiments, the self-contained imaging system 40 and the self-contained light source 30 are in an opposed axial relationship where the self-contained imaging system 40 is configured to be directed in a direction towards the self-contained light source 30. In other embodiments, the self-contained imaging system 40 and the self-contained light source 30 are in a parallel relationship and re generally configured to be directed in the same direction.

During use, the imaging system 40 is pivotably movable to the first, proximal-viewing direction so that an image of the inner space 14 of the sheath 12 is visible when the branch B is being severed from the vessel V. The imaging system 40 is pivotably movable to the second, distal-viewing direction so that an image through the tip 20 is visible when the vessel V is being harvested and separated from the surrounding tissue. In one non-limiting embodiment, the camera 41 is pivotably movable in an accurate manner along a longitudinal axis defined by the sheath 12. In certain embodiments, the camera 41 is pivotably movable in an arc from about 0° to about 180°.

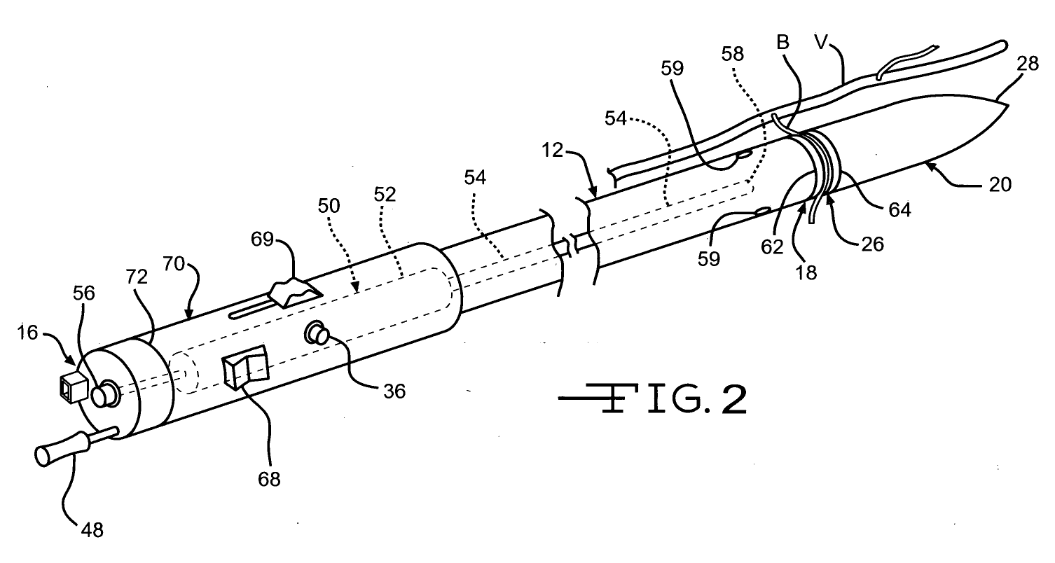

In certain embodiments, the self-contained dissector/harvester device 10 further includes a self-contained insufflation device 50. The insufflation device 50 is axially positioned within the inner space 14 of the sheath 12. The insufflation device 50 includes a cartridge 52 containing a supply of a suitable gas and a supply line 54 having a discharge end 58. In certain embodiments, the discharge end 58 of the supply line 54 is located near the distal end 18 of the sheath 12. The insufflation device 50 can be operatively connected to a release mechanism 56, as shown in FIGS. 1 and 2, to allow the gas to be discharged from the supply line 54. The sheath 12 can include one or more discharge ports or holes 59 that are in communication with the inner space 14. The gas escaping from the ports 59 enters the forming cavity and keeps the surrounding tissue away from the self-contained dissector/harvester device 10. The self-contained insufflation device 50 allows gas to be delivered via the radially extending openings 59 in the distal end 18 of the sheath 14. The gas is delivered in a suitable manner subcutaneously to an area adjacent to the vessel V to be dissected and harvested.

Also, in certain embodiments, the self-contained dissector/harvester device 10 further includes a self-contained severing device 60. In one embodiment, the self-contained severing device 60 can comprise a bipolar electrocautery tool or an ultrasonic cauterizing tool. In certain embodiments, the self-contained severing device 60 comprises a cauterizing tool where a first cauterizing member 62 is operatively mounted on the distal end 18 of the sheath 12 and a second cauterizing member 64 is operatively mounted on the proximal end 26 of the tip 20.

The self-contained severing device 60 is activated by axially displacing the tip 20 in a direction away from the sheath 12. The self-contained severing device 60 is maneuvered adjacent to the branch B such that the branch B is positioned between the distal end 18 of the sheath 12 and the proximal end 26 of the tip 20. The tip 20 is then retracted to a position adjacent to the sheath 12.

The self-contained severing device 60 is activated by axially moving the tip 20 in an axial direction toward the sheath 12. The electrodes 62 and 64 are axially moved toward each other and are used to initially grasp the branch B being dissected. The electrodes 62 and 64 are activated, severing the branch B from the vessel V.

In certain embodiments, the self-contained severing device 60 also includes an axially displacing member 66 that is operatively connected to the tip 20 to move the tip 20 in the axial direction toward and away from the sheath 12. The displacing member 66 can be connected to a displacing button 68 that advances and returns along the longitudinal direction. The advancing and returning force is transmitted to the displacing member 66 and the tip 20 is longitudinally moved.

Also, in certain embodiments, the proximal end 16 of the sheath 12 is operatively connected to a handle 70. The handle 70 can include a removable closure device 72 so that one or more of the self-contained light source 30, self-contained imaging system 40 and self-contained insufflation device 50 can be removed from the inner space 14 of the sheath 12.

To begin the dissection procedure, the dissector tip 20 is inserted through an initial incision in the patient. In operation, the dissector tip 20 is pressed into the tissues surrounding the vessel, thereby forming a tunnel or cavity around the vessel. In certain embodiments, it is desired that the dissector tip 20 be pressed into the surrounding tissue generally along the direction of the vessel in order to separate the vessel from adjacent tissue without damage to the surrounding tissue.

Upon inserting the self-contained dissector/harvester device 10 under the patient's skin, it is possible to obtain an image illuminated by the illuminating light 34 from the self-contained light source 30. The camera 41 in the imaging system 40 can be pivotably moved to the distal-viewing direction such that the vessel is viewed through the transparent tip 20.

In certain embodiments, the self-contained insufflation device 50 is activated for inflating the area adjacent the vessel as the cavity is being formed.

The dissector tip 20 is used to perform an initial, or blunt, dissection of the vessel from the surrounding tissue. Also, the self-contained severing device 60 is engaged to sever any branches B extending from the vessel.

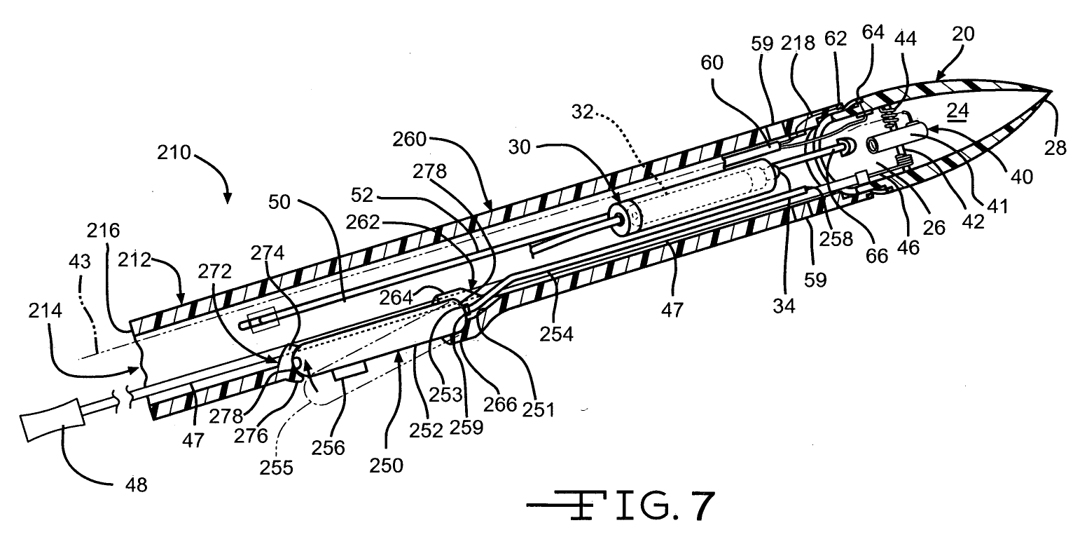

In certain other embodiments, as shown in FIG. 6 for example, the self-contained dissector/harvester device 10 further includes a free-standing insufflation device 150. The insufflation device 150 includes a cartridge 152 containing a supply of a suitable gas and a supply line 154 having a discharge end 158. The discharge end 158 of the supply line 154 is axially positioned within the inner space 14 of the sheath 12 near the distal end 18 of the sheath 12. The supply line 154 also has a proximal end 156 that extends from an opening 15 in the sheath 12. While the opening 151 is shown as being situated on a sidewall of the sheath 12, the opening 151 can be situated at any suitable location on the sheath 12, including by way of a non-limiting example, at the proximal end 16 of the sheath 12. The supply line 154 has a proximal end 159 that extends through the opening 151 and is connected to the cartridge 152. The insufflation device 150 can also include a release mechanism 156 to allow the gas to be discharged from the supply line 154.

In certain other embodiments, as shown in FIG. 7 for example, a self-contained dissector/harvester device 10 further includes a sheath 212 having an externally mounted insufflation device 250. For ease of explanation, features that are the same as in the earlier described embodiments have been given the same reference numerals and are not again described in detail with respect to the embodiment shown in FIG. 7.

The insufflation device 250 includes a cartridge 252 containing a supply of a suitable gas and a supply line 254 having a discharge end 258. The discharge end 258 of the supply line 254 is axially positioned within an inner space 214 of the sheath 212 near a distal end 218 of the sheath 212.

The supply line 254 also has a proximal end 256 that extends through an opening 251 in the sheath 212. The supply line 254 has a proximal end 259 that extends through the opening 251 and is operatively connected to the cartridge 152. The insufflation device 150 can also include a release mechanism 256 to allow the gas to be discharged from the supply line 254.

In the embodiment shown in FIG. 7, the cartridge 252 is mounted on a sidewall 260 of the sheath 212. While the opening 251 is shown as being situated on the sidewall 260 of the sheath 212, the opening 251 can be situated at any suitable location on the sheath 212, including by way of a non-limiting example, at a proximal end 216 of the sheath 212. The sidewall 260 defines a distal detent 262 that includes an inner distal flange 264 that is at least partially within the inner space 214 of the sheath 212, and an outer distal flange 266 that is at least partially extended in a radially outward direction from a plane formed by the sidewall 260. The inner and outer distal flanges 264 and 266, respectively, define a distal recess 278 that is configured to receive at least a first end 253 of the cartridge 252. The recess 278 also has a suitable configuration that allows the proximal end 259 of the supply line 254 to be readily connected to a new cartridge, if needed. In such embodiments, the proximal end 259 comprises a suitable connection member that can allow for quick and easy manipulation of any such new cartridge.

The sidewall 260 also defines a proximal detent 272 that includes an inner proximal flange 274 that is at least partially within the inner space 214 of the sheath 212, and an outer distal flange 276 that at least partially extends in a radially outward direction from a plane formed by the sidewall 260. The inner and outer distal flanges 274 and 276, respectively, define a proximal recess 278 that is configured to receive at least a second end 255 of the cartridge 252. The recess 278 also has a suitable configuration that allows the proximal end 255 of the cartridge 252 to be readily mounted on the sheath 212. In one non-limiting embodiment, as shown in FIG. 7, the outer proximal flange 276 is configured to allow the proximal end 255 of the cartridge 252 to be quickly and readily snapped into the proximal recess 278 so that there can be a quick and easy replacement of the cartridge 252, if needed.

In another particular embodiment, the dissector/harvester device can include a sheath having a severing device therein, a free-standing light source having tether that is externally tethered to a power supply, and a free-standing imaging system having a tether that is externally tethered to an image receiving system. In certain embodiments, the dissector/harvester device further includes a insufflation device to supply a gas subcutaneously to an area adjacent to the vessel. In a particular embodiment, the insufflation device can include a tether that is externally connected to a supply of gas. In another particular embodiment, at least a supply of the gas of the insufflation device can be externally mounted on the sheath.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

What is claimed is:

1. A device for dissecting and/or harvesting a vessel and severing any branches extending therefrom comprises:
a sheath configured to be at least partially inserted in a body through a cut skin portion;
a self-contained light source positioned within the sheath;
a self-contained imaging system positioned within the sheath; and,
a severing device positioned within the sheath and configured to sever the vessel and branches.

2. The dissector/harvester device of claim 1, further including an insufflation device configured to supply a gas subcutaneously to an area adjacent to the vessel.

3. The dissector/harvester device of claim 1, wherein the severing device comprises a tip in an axial position over a distal end of the sheath, the tip being configured to be axially movable between an open position and a closed position configured to dissect the vessel and branch upon movement to the closed position.

4. The dissector/harvester device of claim 1, wherein the severing device is configured to cauterize the severed branches and vessel.

5. The dissector/harvester device of claim 1, wherein the self-contained imaging system and the self-contained light source are in an opposed axial relationship, whereby the self-contained imaging system is positioned to receive images in an area adjacent to the self-contained light source.

6. The dissector/harvester device of claim 3, wherein the sheath is substantially stationary with respect to the tip.

7. The dissector/harvester device of claim 1, wherein the light source includes an LED or optical fiber.

8. The dissector/harvester device of claim 1, wherein the light source includes a battery-operated power supply.

9. The dissector/harvester device of claim 1, wherein the self-contained imaging system is configured to be pivotably movable within the sheath.

10. The dissector/harvester device of claim 1, wherein the self-contained imaging system comprises a wireless camera.

11. The dissector/harvester device of claim 3, wherein the tip has a distal end configured to dissect the vessel from surrounding tissue.

12. The dissector/harvester device of claim 1, wherein the severing device comprises an ultrasonic cauterizing tool or a bipolar electrocautery tool.

13. The dissector/harvester device of claim 1, wherein the sheath includes at least one removable closure device at a proximal end thereof.

14. The dissector/harvester device of claim 1, wherein one or more of the self-contained light source and the self-contained imaging system are removable from the device.

15. The dissector/harvester device of claim 1, wherein the insufflation device is removable from the device.

16. The dissector/harvester device of claim 3, wherein the distal end of the sheath and a proximal end of the tip each has at least one generally mating surface whereby, when the tip is adjacent to the sheath, the generally mating surfaces are configured to secure the vessel being harvested while a vessel branch is being severed.

17. The dissector/harvester device of claim 3, wherein the tip and the sheath include electrodes configured for being electrically energized to sever and cauterize a vessel branch.

18. The dissector/harvester device of claim 1, further including an insufflation device configured to supply a gas subcutaneously to an area adjacent to the vessel.

19. The dissector/harvester device of claim 18, wherein the insufflation device is at least partially positioned within the sheath, and is removable therefrom.

20. The dissector/harvester device of claim 18, wherein the insufflation device is axially positioned within the sheath, and is removable therefrom

21. A method of severing branches from a vessel during harvesting from a body by a self-contained dissector/harvester device, the self-contained dissector/harvester device having:
a sheath configured to be inserted in a body through a cut skin portion;
a self-contained light source positioned within the sheath;
a self-contained imaging system positioned within the sheath; and,
a severing device positioned within the sheath and configured to sever the vessel and branches;
the method comprising the steps of:
inserting a distal end of the self-contained dissector/harvester device into the body alongside the vessel to form a cavity substantially surrounding the vessel;
activating the self-contained light source to at least illuminate a portion of the branch;
activating the self-contained imaging system to view at least a portion of the illuminated branch;
moving the severing device to a position substantially adjacent to the branch and activating the severing device, whereby the vessel and branch are severed from the vessel.

22. The method of claim 21, wherein the self-contained dissector/harvester device further includes an insufflation device, wherein the method further includes supplying a gas subcutaneously to an area adjacent to the vessel to be dissected and harvested.

23. The method of claim 21, in which one or more of the self-contained light source and self-contained imaging system and/or insufflation devices can be removed and replaced.

24. The method of claim 21, in which the insufflation device can be removed and replaced.
